(19) United States
(12) Patent Application Publication
(10) Pub. No.: US 2007/0184484 A1
(43) Pub. Date: Aug. 9, 2007

(54) Anti-abnormal type prion monoclonal antibody, process for producing the same, and immunoassay of abnormal type prion protein using the same

(75) Inventors: Morikazu Shinagawa, Tsukuba-shi (JP); Motohiro Horiuchi, Obihiro-shi (JP); Atsushi Umetani, Chuo-ku (JP)

Correspondence Address: BIRCH STEWART KOLASCH & BIRCH, PO BOX 747, FALLS CHURCH, VA, 22040-0747, US

(73) Assignees: Obihiro University of Agriculture and Veterinary Medicine, Obihiro-shi (JP); Fujirebio Inc., Tokyo (JP)

(21) Appl. No.: 10/512,981

(22) PCT Filed: Oct. 31, 2002
(86) PCT No.: PCT/JP02/11347

§ 371 Date: Feb. 20, 2007

(30) Foreign Application Priority Data
Apr. 30, 2002 (JP) 2002-129003

(51) Int. Cl. G01N 33/53; C12N 5/06; C07K 16/40

(52) U.S. Cl. 435/7.1; 530/388.26; 435/327

(57) ABSTRACT

A monoclonal antibody which recognizes abnormal type prion protein alone is disclosed. By the present invention, an anti-abnormal type prion monoclonal antibody whose corresponding antigen is an abnormal type prion protein, and which does not substantially react with normal type prion protein was first provided. Since the corresponding antigen of the monoclonal antibody according to the present invention is the naturally occurring abnormal type prion protein, measurement of the abnormal type prion protein may be attained with high sensitivity.

### DESCRIPTION

Anti-abnormal Type Prion Monoclonal Antibody, Process for Producing the Same, and Immunoassay of Abnormal Type Prion Protein Using the Same

1. Technical Field

The present invention relates to an anti-abnormal type prion monoclonal antibody, process for producing the same, and immunoassay of abnormal type prion using the same.

2. Background Art

Cranial nervous diseases including Creutzfeldt-Jakob disease (hereinafter referred to as “CJD” for short), scrapie disease of sheep, transmissible encephalopathy of mink and the like develop after a long incubation period. They are mainly characterized by spongiform change of brain tissue and amyloid spot (kuru spot), that are almost localized in nerve system, and progressively aggravate to death. Although the cause of the diseases has not been fully clarified, the so called “prion hypothesis” which assumes that the diseases are not caused by infectious pathogen such as a virus, but are caused by deposition of abnormal type prion protein, is now believed by most of the researchers. These diseases are diagnosed by pathological analysis of thin section of brain tissue.

It is thought that these diseases are infectious, and infection is made by eating cranial nerve tissue (e.g., kuru disease and the like), or by medical treatment such as piercing of electrodes or transplantation of endocranium. Recently, it is thought that bovine spongiform encephalopathy and new type CJD are infected by oral infection.

Normal prion protein is a glycoprotein existing in cell membranes, which widely occurs in various eukaryotes such as yeasts. The gene encoding normal prion is a single gene and the encoded amino acid sequence is very well conserved among the mammals. Especially, it has been reported that the homology of the amino acid sequences among human, sheep and bovine is not less than about 90%.

Although the function of the normal type prion protein has not yet been clarified, since the amino acid sequence is well conserved, it is presumed that it plays an important role in generation, development and function of nerve tissue. With a knock out mouse in which the prion gene is knocked out, abnormal walking such as shaking of the lower half of the body with aging, and pathologically, atrophy of cerebellum, especially deciduation of cerebellar Purkinje cells, is observed.

In general, it is said that there is no difference between the amino acid sequences of prion protein in the individuals suffering from prion disease and in normal individuals. Therefore, it is thought that deposition of the abnormal prion protein is not because of the amino acid sequence, but because of the difference in stereostructure. Therefore, the conventional anti-prion antibodies which recognize the primary amino acid sequence of prion cannot distinguish the abnormal type prion from the normal type prion. Further, since the amino acid sequence is well conserved between animals, it is presumed that antigenecity of prion is low. Thus, production of an anti-abnormal type prion antibody using an immunogen keeping the stereostructure thereof, in which the antigenecity of the immunogen is increased, or production of an animal to be immunized, which recognizes the antigenecity is demanded.

### SUMMARY OF THE INVENTION

An object of the present invention is to provide a monoclonal antibody which recognizes the abnormal type prion alone, as well as a production process thereof. Another object of the present invention is to provide an immunoassay of the abnormal type prion using the monoclonal antibody.

It is thought that a monoclonal antibody which recognizes the abnormal type prion alone may be obtained by immunizing an animal with the abnormal type prion, obtaining monoclonal antibodies by a conventional method, and by screening a monoclonal antibody which reacts with the abnormal type prion but does not react with the normal type prion. However, not only because of the fact that there are no differences in the amino acid sequence of the normal and abnormal types, but also because of the low species specificity, it is difficult to induce an antibody, especially an antibody which can distinguish the abnormal type prion from the normal type prion.

As a result of intensive study, the present inventors succeeded in preparing a monoclonal antibody which recognizes abnormal type prion protein alone, by using an abnormal type prion protein which retains the stereostructure of the abnormal prion protein as well as possible, and by immunizing a PrP gene-deficient mouse with the immunogen, thereby completing the present invention.

That is, the present invention provides an anti-abnormal type prion monoclonal antibody whose corresponding antigen is an abnormal type prion protein, and which does not substantially react with normal type prion protein, or an antigen-binding fragment thereof. The present invention also provides a monoclonal antibody which undergoes antigen-antibody reaction with an abnormal type prion protein treated with proteinase K having a final concentration of 80 μg/ml, or an antigen-binding fragment thereof. The present invention further provides the monoclonal antibody produced by hybridoma 6H10 (FERM BP-8226), or an antigen-binding fragment thereof. The present invention still further provides a hybridoma which produces the monoclonal antibody according to the present invention. The present invention still further provides a method for measuring an abnormal type prion by an immunoassay utilizing antigen-antibody reaction between the monoclonal antibody or the antigen-binding fragment thereof according to the present invention and the abnormal type prion. The present invention still further provides a kit for immunoassay, comprising the monoclonal antibody or the antigen-binding fragment thereof according to the present invention. The present invention still further provides a method for producing the monoclonal antibody according to the present invention, comprising immunizing an animal with an abnormal type prion protein; preparing hybridomas originated from antibody-producing cells of the immunized animal; screening a hybridoma producing an anti-abnormal type prion monoclonal antibody that reacts with non-denatured abnormal type prion protein but does not react with normal type prion protein; and recovering the anti-abnormal type prion monoclonal antibody from the hybridoma selected by the screening.

By the present invention, an anti-abnormal type prion monoclonal antibody whose corresponding antigen is an abnormal type prion protein, and which does not substantially react with normal type prion protein, was first provided. Since the corresponding antigen of the monoclonal antibody according to the present invention is the naturally occurring abnormal type prion protein, measurement of the abnormal type prion protein may be attained with high sensitivity. Therefore, it is thought that the present invention will greatly contribute to the diagnosis of prion disease.

### BRIEF DESCRIPTION OF THE DRAWINGS

FIG. 1 shows the reactivities between abnormal type prion protein treated with various concentrations of proteinase K and various monoclonal antibodies, which were tested by ELISA; and

FIG. 2 shows the reactivities between abnormal type prion protein treated with various concentrations of guanidine hydrochloride after treatment with proteinase K and various monoclonal antibodies, which were tested by ELISA.

### BEST MODE FOR CARRYING OUT THE INVENTION

The monoclonal antibody according to the present invention is one whose corresponding antigen is an abnormal type prion protein, and which does not substantially react with the normal type prion protein. The term “whose corresponding antigen is an abnormal type prion protein” means that the monoclonal antibody is originated from an animal immunized with the abnormal type prion protein as an immunogen. However, a monoclonal antibody prepared by another method, such as genetic engineering method, is also the monoclonal antibody of the present invention if the monoclonal antibody is the same as the monoclonal antibody whose corresponding antigen is the abnormal type prion protein. The term “does not substantially react with normal type prion protein” means that the reactivity with the normal type prion is lower than the reactivity with the abnormal type prion to a discernable degree. Therefore, even in cases where a monoclonal antibody has a cross-reactivity with the normal type prion, if the immunological reactivity is lower than the immunological reactivity with the abnormal type prion to a discernable degree, the monoclonal antibody is included in the case where it “does not substantially react with normal type prion protein”, and so the monoclonal antibody is included within the scope of the present invention. Needless to say, a monoclonal antibody which does not have cross-reactivity with the normal type prion, that is, a monoclonal antibody which reacts with the abnormal type prion but not with the normal type prion is preferred.

The monoclonal antibody according to the present invention is preferably one whose corresponding antigen is a non-denatured abnormal type prion, that is, an abnormal type prion which was not denatured by a protein-denaturant such as guanidine hydrochloride. The monoclonal antibody is also preferably one which does not react with an abnormal type prion protein solubilized by a denaturant such as guanidine hydrochloride. Further, the monoclonal antibody of the present invention is preferably one which undergoes antigen-antibody reaction with an abnormal type prion protein treated with proteinase K having a final concentration of 80 λg/ml. Such a monoclonal antibody recognizes the stereostructure unique to the abnormal type prion protein, so that it can clearly distinguish between the abnormal type and normal type. An example of such an anti-abnormal type prion monoclonal antibody is the monoclonal antibody produced by hybridoma 6H10 prepared in the Example below. The hybridoma 6H10 has been deposited with intemational Patent Organism Depositary, National Institute of Advanced Industrial Science and Technology, AIST Tsukuba Central 6, 1-1-1 Higashi, Tsukuba, Ibaraki, Japan as from Apr. 11, 2002 under an accession number FERM P-18821, and the deposition was converted to international deposition under the Budapest Treaty on Oct. 28, 2002 under an accession number FERM BP-8226.

The present invention also provides antigen-binding fragments of the above-described monoclonal antibody according to the present invention. The term “antigen-binding fragment” herein means fragment such as Fab fragment or F(ab′)₂ fragment of the antibody, which exhibits antigen-binding property of the antibody.

These fragments may easily be obtained by cleaving the monoclonal antibody of the present invention with papain or pepsin according to a conventional method. These antigen-binding fragments may be used in the immunoassays described below equally as the monoclonal antibody of the present invention.

As mentioned above, it is difficult to prepare a monoclonal antibody which reacts with the abnormal type prion protein but does not substantially react with the recombinant prion protein by using the abnormal type prion as an immunogen. To solve this problem, the present inventors immunized a PrP gene-deficient mouse with the abnormal type mouse prion protein separated from the brain of an abnormal type prion-infected mouse of the same species, with retaining the stereostructure of the abnormal type prion protein as well as possible, so as to obtain a monoclonal antibody which specifically reacts with the abnormal type prion protein. The abnormal type prion protein used as the immunogen is preferably one separated by a physical separation method such as separative centrifugation. Preferred conditions for the separative centrifugation method are described in detail in the Example below.

By purifying the immunogen avoiding the chemical treatment such as treatment with a denaturant as much as possible, the monoclonal antibody of the present invention may be more likely obtained.

The monoclonal antibody according to the present invention may be prepared by a conventional method except that the above-described immunogen and the above-described animal to be immunized are used. That is, the animal is immunized with the above-described immunogen, and hybridomas derived from antibody-producing cells of the immunized animal are prepared. The hybridomas are screened for those producing monoclonal antibodies which react with the abnormal type prion protein and does not substantially react with the recombinant prion protein, and the desired monoclonal antibody is recovered from the screened hybridoma. The method for preparing hybridomas by fusing antibody-producing cells such as spleen cells and lymphocytes with immortalized cells such as myeloma cells is well-known in the art.

By an immunoassay utilizing the antigen-antibody reaction between the monoclonal antibody according to the present invention and the abnormal type prion, the abnormal type prion may be measured. The term “measure” herein includes both detection and quantification. The immunoassays per se are well-known in the art, and any of the well-known immunoassays may be employed in the present invention. That is, classifying the known immunoassays according to the reaction type, known immunoassays include sandwich immunoassays, competition immunoassays and agglutination immunoassays. Classifying the known immunoassays according to the label employed, known immunoassays include enzyme immunoassays, radio immunoassays, fluorescence immunoassays and the like. Any of these immunoassays are included in the “immunoassay” defined in the present invention. Further, immunohistostaining, Western blotting and the like are also included in the “immunoassay” defined in the present invention.

These immunoassays per se are well-known in the art, and so it is not necessary to explain these immunoassays in the present specification. Briefly, in sandwich immunoassays, for example, the monoclonal antibody or an antigen-binding fragment thereof is immobilized on a solid support as a primary antibody. The primary antibody is then reacted with a sample, and after washing the solid support, the resultant is then reacted with a secondary antibody which reacts with the abnormal type prion by antigen-antibody reaction (the secondary antibody may be an antibody which also reacts with the normal type prion, and may be either a monoclonal antibody or a polyclonal antibody). After washing the solid support, the secondary antibody bound to the solid support is measured. By labeling the secondary antibody with an enzyme, fluorescent substance, radioactive substance, biotin or the like, measurement of the secondary antibody may be attained by measuring the label. The above-mentioned measurement is conducted for a plurality of standard samples each containing a known concentration of the abnormal type prion, and the relationship between the concentrations of the abnormal type prion in the standard samples and the measured amounts of the label is plotted to prepare a calibration curve. The abnormal type prion in a test sample may be determined by applying the measured amount to the calibration curve. It should be noted that the above-mentioned primary antibody and the above-mentioned secondary antibody may be exchanged. In agglutination immunoassays, the monoclonal antibody according to the present invention or an antigen-binding fragment thereof is immobilized on particles such as latex particles, and the particles are reacted with a sample, followed by measurement of the absorbance. The above-mentioned measurement is conducted for a plurality of standard samples each containing a known concentration of the abnormal type prion, and the relationship between the concentrations of the abnormal type prion in the standard samples and the measured absorbance is plotted to prepare a calibration curve. The abnormal type prion in a test sample may be determined by applying the measured absorbance to the calibration curve.

The reagents necessary for each type of immunoassay are also well-known in the art. Except for the monoclonal antibody used, the immunoassay according to the present invention may be carried out using an ordinary kit for immunoassay. For example, such an immunoassay kit may usually include buffer solution, solid support, labeled secondary antibody and the like.

### EXAMPLE

The present invention will now be described in more detail by way of examples thereof. It should be noted that the present invention is not restricted to the examples below.

(1) Preparation Of Immunogen

Mouse abnormal type prion protein (PrPSc) purified from prion-infected mouse brain by separative centrifugation method was used as an immunogen. The purification of the mouse abnormal type prion protein was carried out by the following method:
1) collecting infected brain tissue and cutting the tissue into pieces of appropriate size;
2) washing the pieces twice with PBS;
3) homogenizing the resultant after adding a homogenation buffer (brain tissue/homogenation buffer=1:9; 10% brain emulsion);
4) centrifuging the homogenate at 15,000 rpm for 30 minutes at 4° C., and collecting the supernatant;
5) ultracentrifuging the supernatant at 45,000 rpm for 3 hours at 4° C., and collecting the precipitate;
6) adding buffer B to the precipitate and homogenizing the precipitate;
7) ultracentrifuging the homogenate at 45,000 rpm for 3 hours at 4° C.;
8) adding buffer C to the precipitate and homogenizing the precipitate;
9) adding RNase A to the solution to a final concentration of 100 μg/ml, and allowing reaction at room temperature for 30 minutes and then at 4° C. overnight;
10) overlaying the resulting sample on 1 M sucrose cushion, ultracentrifuging the resultant at 45,000 rpm for 2 hours at 20° C., and collecting the precipitate;
11) suspending the precipitate in 0.1 % ZWITTERGENT (zwitterionic surfactant produced by CALBIOCHEM)-PBS;
12) homogenizing the solution;
13) ultracentrifuging the solution at 25,000 rpm for 20 minutes at 4° C., and collecting the precipitate; and
14) suspending the precipitate in 0.1% ZWITTERGENT-PBS to obtain the immunogen.
   Used Buffers
1) homogenation buffer: 10% Sarkosyl, 10 mM Tris-HCl, 133 mM NaCl, 1 mM EDTA, 1 mM DTT, pH8.3
2) Buffer B : 10 mM Tris-HCl, 1.71 M NaCl, 1 mM EDTA, 1% Sarkosyl, 1 mM DTT, pH8.3
3) Buffer C: 10 mM Tris-HCl, 100 mM NaCl, 5 mM MgCl₂, pH7.4
4) Sucrose cushion: 1 M sucrose, 100 mM NaCl, 0.5% ZWITTERGENT, 10 mM PB, pH6.9
   (2) Immunization and Cell Fusion

The purified mouse PrPSc (concentration: 1.0 mg/ml) obtained in (1) was mixed with equivolume of Freund's complete adjuvant, and the resultant was subcutaneously administered to PrP gene-deficient mice (Yokoyama et al., Journal of Biological Chemistry, vol. 276, 11265-11271, 2001) at the cervical part at a concentration of 0.2 mg/ml. The immunization was carried out totally three times with two weeks' interval. Three days after the final immunization, the spleen was recovered from each mouse and the cells were dispersed. The cells were fused with P3U1 myeloma cells by polyethylene glycol method.

Hybridomas were selected by culture in HAT medium, and the screening of the antibody-producing cells was carried out by ELISA using the above-described purified mouse PrPSc antigen. That is, each cell culture supernatant was placed in a well of an ELISA plate (96 well-type) in which the purified mouse PrPSc antigen obtained in (1) was immobilized. After reaction and washing, horse radish peroxidase (HRP)-labeled anti-mouse Ig (IgG+IgM) was reacted and existence of antibody-producing cell was checked based on the coloring reaction. Further, ELISA was carried out in the similar manner using a known recombinant normal type prion (Simone Homemann et al., “Recombinant full-length murine prion protein, mPrP(23-231):purification and spectroscopic characterization”, Federation of European Biochemical Societies (FEBS) Letters 413 (1997) 277-281) as the antigen, and hybridomas each producing a monoclonal antibody which did not undergo antigen-antibody reaction with the recombinant normal type prion were selected.

As a result, as hybridomas each producing an anti-abnormal type prion protein monoclonal antibody, 6H10, 31C6, 72-5 and 44B1 were obtained. Among these, the monoclonal antibody produced by 31 C6, 72-5 or 44B1 underwent antigen-antibody reaction with the recombinant normal type prion, but the monoclonal antibody produced by 6H10 did not undergo antigen-antibody reaction with the recombinant normal type prion. Therefore, the monoclonal antibody (mAb 6H10) produced by 6H10 is the monoclonal antibody according to the present invention. As mentioned above, hybridoma 6H10 has been deposited with International Patent Organism Depositary, National Institute of Advanced Industrial Science and Technology under the accession No. FERM BP-8226.

(3) Analysis of Reactivity of Monoclonal Antibody Specifically Reacting with Abnormal Type Prion Protein

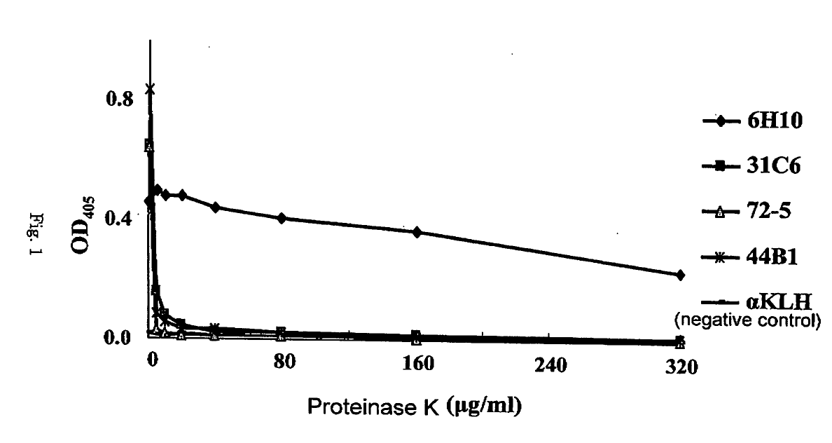

Analysis of the reactivity with PrPSc was carried out by ELISA using immobilized purified PrPSc. After adsorbing the PrPSc fraction on an ELISA plate, the adsorbed PrPSc was treated with proteinase K (PK) at various final concentrations (0, 10, 20, 40, 80, 160 or 320 μg/ml). With increase in the PK concentration, while the mAb 31 C6, 72-5 and 44B1 reacting with both PrPc and PrPSc more and more hardly reacted with the PK-treated PrPSc, mAb 6H10 reacted with the PrPSc fraction even after the PK digestion at 320 μg/mI (FIG. 1). This indicates that what is recognized by mAb 6H10 is not PrPc. The conditions for immobilizing the purified PrPSc, the conditions of the PK treatment, and concrete method of the ELISA were as follows:
1) Preparation of PrPSc-adsorbed Plate

Purified PrPSc is placed into wells at 200 ng/50 μ1/well (in 20 mM phosphate buffer), and allowed to react at 4° C. overnight.
2) Conditions of PK Treatment (buffer, temperature, time and so on)

To the PrPSc-immobilized plate, PK diluted to various concentrations (final concentration of 0, 5, 10, 20, 40, 80, 160 or 320 μg/ml, in 50 mM Tris/HCl, pH8.0/150 mM NaCl) is added, and the resultant is allowed to react at 37° C. for 45 minutes.
3) Termination of PK Reaction and Blocking of Plate

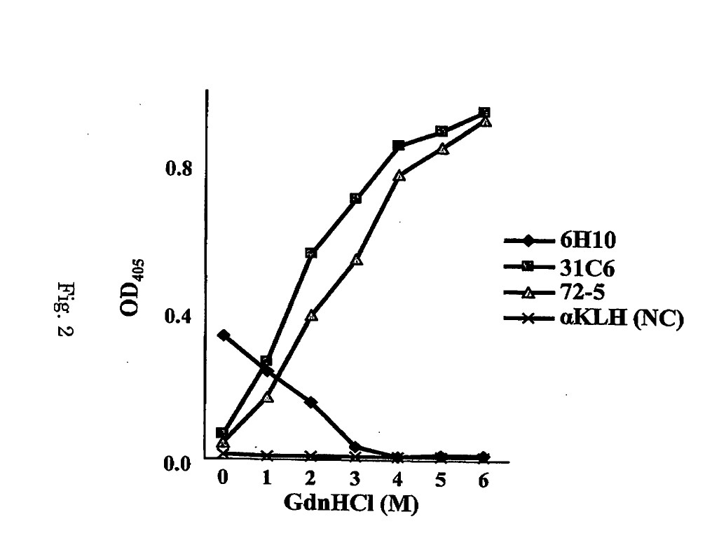

After the PK treatment, the PK activity is inhibited (2 mM) by Pefabloc (ROCHE), and then blocking is carried out with 5% FBS-PBST (room temperature for 2 hours).
4) After the blocking, the plate is washed three times with PBST, primary antibody (various MAbs) is added (50 μl/well), and the resultant is allowed to react at room temperature for 1 hour.
5) After the reaction with the primary antibody, the plate is washed three times with PBST, secondary antibody (anti-mouse immunoglobulin rabbit antibody (produced by AMERSHAM) is added (50 μl/well), and the resultant is allowed to react at room temperature for 1 hour.
6) After the reaction, the plate is washed three times with PBST, a substrate and a coloring agent (ABTS) are added to the wells, and the resultant is allowed to react at room temperature for 30 minutes.
7) After the enzyme reaction, absorbance of the solutions in the plate is measured by using a plate reader.
(ii) After adsorbing PrPSc on a plate and after PK digestion (concentration: 40 μg/ml) in the similar manner as described above, PrPSc was treated with guanidine hydrochloride (GdnHCI) at various final concentrations (0, 1, 2, 3, 4, 5 or 6 M), and reactivity with each monoclonal antibody was checked by ELISA in the same manner as described above. The reactivity with mAb 6H10 disappeared at a GdnHCI concentration of 3M, while the reactivities of mAbs 31C6 and 72-5 which react with both PrPc and PrPSc proportionally increased with the GdnHCI concentration. This suggest the possibility that mAb 6H10 recognizes the structure of PrPSc (FIG. 2).
(iii) In another method, mAb 6H10 did not react with PrPSc by Western Blotting using scrapie-infected mouse brain emulsion. This is coincident with the above-described result that mAb 6H10 does not recognize PrPSc when the structure of PrPSc is changed. The method for preparing the mouse brain emulsion and the concrete conditions of WB were as follows:
   Preparation of Brain Emulsion (In Case Of Using Mixer Mill Of QUIAGEN)
1) In a 2 ml ASSIST's tube with round base, 200 mg of brain tissue is placed.
2) To the tube, 800 μl of TN buffer and one tungsten bead are added.
3) The mixture is shaken by a mixer mill at 20 Hz for 45 seconds.
4) The resultant is prepared to 20%(W/W) emulsion and stored in a tube with 0-ring.
   Concrete Conditions Of WB
   Sample Preparation
1) To 250 μl of the 20%(W/W) brain emulsion, 250 μl of surfactant buffer is added and the mixture is subjected to vortex and ultrasonication treatments.
2) To the resultant, 20 μl of 1 mg/ml PK is added, and digestion reaction is allowed to occur at 37° C. for 30 minutes (in water bath).
3) To the resultant, 10 μl of Pefablock (ROCHE) is added and the mixture is stirred (Vortex) so as to terminate the enzyme reaction.
4) To the resultant, 250 μl of butanol-methanol solution is added and stirred (Vortex).
5) The mixture is centrifuged at 15,000 rpm for 10 minutes at 20° C., and the precipitate is lightly dried.
6) To the precipitate, 100 μl of 1 x sample buffer is added and the mixture is boiled at 100° C. for 5 minutes. In cases where the precipitate is hardly dissolved, ultrasonication is performed.
   SDS-PAGE

Precast gel of INVITROGEN (formerly NOVEX) is used.
Gel: NuPAGE 12% Bis-Tris Gel, 1.0 mm, 12 well (no. NP0342)
Buffer: antioxidant (No. NP0005)-containing NuPAGE MOPS SDS running buffer (No. NP0001)
Conditions of electrophoresis are in accordance with the instruction by INVITROGEN.

The amount of sample is 20 μl (equivalent to 10 mg of tissue) Western Blot
Wet type blotting apparatus is used.
Buffer: the buffer described in the Instruction by INVITROGEN (NuPAGE transfer buffer (No. NP0006), antioxidant (No. NP0005) and methanol) to which SDS is added to a concentration of 0.01%.

The conditions for blotting were 40V constant voltage for 4 to 15 hours.

Immunostaining

1) The membrane after the blotting is transferred to a cylindrical vessel, a blocking agent (PBST supplemented with 5% skim milk) is added thereto, and the reaction is allowed to occur on a membrane roller while rotating the cylindrical vessel at room temperature for 1 hour.
2) After the blocking, 10 ml of the primary antibody solution (B103 anti-PrP antibody and the like: 0.1-10 μg/ml; PBST supplemented with 1% skim milk) is added, and the reaction is allowed to occur on the membrane roller at room temperature for 1 hour.
3) The resultant is washed with PBST for 20 minutes. During this washing, PBST is replaced with fresh one 5times.
4) To the resultant, 10 ml of the secondary antibody solution (AMERSHAM NA9340: diluted to 1:2500 with PBST supplemented with 1% skim milk) is added, and the reaction is allowed to occur on the membrane roller at room temperature for 45 minutes.
5) The resultant is washed with PBST for 20 minutes. During this washing, PBST is replaced with fresh one 5 times.
6) The membrane is removed from the vessel onto a stainless steel vat, ECL (AMERSHAM) is added to conduct luminous reaction.
7) An X-ray film is exposed to the membrane for 2minutes, and the film is developed.
8) During the development step, the next X-ray film is exposed.
9) Thirty minutes later, the films are developed (that is, X-ray films exposed for 2 minutes and for 30 minutes, respectively are developed).
   Reagents Used

TN Buffer:

100 mM NaCl, 50 mM Tris-HCl (pH 7.5) Surfactant Buffer: 4% Zwittergent 3-14, 1% Sarkosyl, 100 mM NaCl, 50 mM Tris-HCI (pH 7.5) butanol-methanol solution: 2-butanol:methanol =5:1 Proteinase K: 1 mg/ml in 50 mM Tris-HCl (pH 8.0), 1 mM CaCi₂, dividedly placed, stored at −20° C. Pefablock: 0.1 M in distilled deionized water (DDW), dividedly placed, stored at −20 ° C.
(iv) In addition, immunoprecipitation was carried out using the PK-digested partially purified PrPSc fraction as an antigen, anti-PrP antibody and protein G-bound immunomagnetic beads. The PrPSc contained in ppt (precipiate) and in sup (supernatant) was detected by biotinylated B-103 anti-PrP antibody (Horiuchi, M., Yamazaki, N., Ikeda, T., Ishiguro, N., and Shinagawa, M., J. Gen. Virol. 76: 2583-2587, 1995) after WB. With mAb 6H10, immunoprecipitation of PrPSc was observed, while with mAb 31C6 or anti-KLH monoclonal antibody (αKLH: mAb serving as a negative control), PrPSc remained in sup. These results indicate that mAb 6H10reacts with the PrPSc aggregations existing in the brain. More concretely, this experiment was carried out as follows:
1) To 100 μl of protein G-bound magnetic beads (DYNAL), 1 ml of a blocking solution (PBST supplemented with 5% skim milk and 50% Sea block, PIERCE) is added, and the mixture is allowed to react at room temperature for 1 hour.
2) After the blocking, 1 ml of a mixture (in PBST supplemented with 1% Triton X-100 (trademark)) of 2 μg of the purified PrPSc fraction and 10 μg of the antibody is added, and the reaction is allowed to occur at 37° C. for 45 minutes.
3) The resulting beads were washed four times with PBST supplemented with 1% Triton X-100, and 100 μl of SDS-PAGE sample buffer is added to conduct elution.
5) Using the eluted SDS-PAGE buffer, WB analysis is carried out by the method described above.
   (4) Immunoassay Using 6H10

As an example of immunoassays using the specific anti-abnormal type prion monoclonal antibody 6H10, agglutination method will now be described.
(i) mAb 6H10 and protein G-bound magnetic beads are mixed and allowed to react at 37° C. for 30 minutes (preparation of antibody-bound magnetic beads).
(ii) Mouse brains from prion disease-infected mouse and from non-infected mouse are treated by the prescribed method (described in the above (3)(iv)) and 10-20% emulsions are prepared.
(iii) The supernatant obtained by centrifuging the emulsion mentioned above (ii) and the antibody-bound beads prepared in above (iii) are mixed, and allowed to react at 37° C. for 30 minutes.
(iv) After the reaction mentioned in (iii) above, the antibody-bound magnetic beads are separated by a magnet, then dispersed again in PBS, and the agglutination thereof is observed. In cases where PrPSc antigen exists in the sample, the magnetic beads are agglutinated and precipitate. On the other hand, in cases where PrPSc does not exist, the magnetic beads are not agglutinated and remain suspended.

By this method, immunoassay was performed using mouse brains infected by two types of abnormal type prion-producing mouse brain cell lines (OBIHIRO strain, (Shinagawa M, Matsuda A, Sato G, Takeuchi M, Ichijo S, Ono T., Nippon Juigaku Zasshi. 1984 Dec;46(6):913-6) and 13/15 strain (Race RE, Fadness LH, Chesebro B.,J Gen Virol. 1987 May;68 (Pt 5):1391-9), respectively, and a normal mouse brain as samples. As a result, the mouse brains infected with the two types of the established cell lines, respectively, showed agglutination, while the normal mouse brain did not show agglutination.

(5) Conclusion

As a result, a monoclonal antibody whose corresponding antigen is the abnormal type prion protein, which does not substantially react with normal type prion protein, and which can distinguish between these prion proteins, was obtained.

1. An anti-abnormal type prion monoclonal antibody whose corresponding antigen is an abnormal type prion protein, and which does not substantially react with normal type prion protein, or an antigen-binding fragment thereof.

2. The monoclonal antibody according to claim 1, whose corresponding antigen is a non-denatured abnormal type prion protein, or an antigen-binding fragment thereof.

3. The monoclonal antibody according to claim 1 or 2, which undergoes antigen-antibody reaction with an abnormal type prion protein treated with proteinase K having a final concentration of 80 μg/ml, or an antigen-binding fragment thereof.

4. A monoclonal antibody which undergoes antigen-antibody reaction with an abnormal type prion protein treated with proteinase K having a final concentration of 80 μg/ml, or an antigen-binding fragment thereof.

5. The monoclonal antibody according to claim 4, which does not undergo antigen-antibody reaction with an abnormal type prion protein denatured by treatment with guanidine hydrochloride having a final concentration of 3M, or an antigen-binding fragment thereof.

6. The monoclonal antibody produced by hybridoma 6H10 (FERM BP-8226), or an antigen-binding fragment thereof.

7. The monoclonal antibody or the antigen-binding fragment thereof according to claim 1, which is a monoclonal antibody.

8. A hybridoma which produces the monoclonal antibody according to claim 1.

9. The hybridoma according to claim 8, which is hybridoma 6H10 (FERM BP-8226).

10. A method for measuring an abnormal type prion by an immunoassay utilizing antigen-antibody reaction between said monoclonal antibody or the antigen-binding fragment thereof according to claim 1, and the abnormal type prion.

11. A kit for immunoassay, comprising said monoclonal antibody or the antigen-binding fragment thereof according to claim 1.

12. A method for producing said monoclonal antibody according to claim 1, comprising immunizing an animal with an abnormal type prion protein; preparing hybridomas originated from antibody-producing cells of said immunized animal; screening a hybridoma producing an anti-abnormal type prion monoclonal antibody that reacts with non-denatured abnormal type prion protein but does not react with normal type prion protein; and recovering said anti-abnormal type prion monoclonal antibody from the hybridoma selected by said screening.

13. The method according to claim 12, wherein said abnormal type prion protein to be administered in the immunization is one separated from brain tissue by separative centrifugation.

14. The method according to claim 12, wherein said animal is a prion gene-deficient animal.
